SEQUENCE LISTING (1 sequence)

SEQ ID NO: 1 - DNA, 52 bp, Artificial Sequence
  RNA which inhibits Lentivirus catalase synthesis
ccggccctcttataccagttggcaactcgagttgccaactggtataagaggg

(19) United States
(12) Patent Application Publication
(10) Pub. No.: US 2012/0107923 A1
(43) Pub. Date: May 3, 2012

(54) EXPRESSION VECTORS WHICH INHIBIT SYNTHESIS OF CATALASE AND USES THEREOF

(76) Inventors: Yedy Israel Jacard, Santiago (CL); Maria Elena Quintanilla Gonzalez, Santiago (CL); Lutske Tampier De Jong, Santiago (CL); Alvaro Diego Bustamante Cadiz, Santiago (CL); Mario Herrera Marschitz, Santiago (CL); Eduardo Karahanian Vartevanian, Santiago (CL)

(21) Appl. No.: 13/197,076

(22) Filed: Aug. 3, 2011

(30) Foreign Application Priority Data
Aug. 6, 2010 (CL) 842-2010

(51) Int. Cl. C12N 15/63

(52) U.S. Cl. 435/320.1

(57) ABSTRACT

Vector and application of a medicine/drug that inhibits the intake of alcohol for prolonged periods, by inhibiting the synthesis of brain catalase or by destroying the product that is generated when brain catalase acts upon ethyl alcohol.

### FIELD OF THE INVENTION

This invention relates to expression vectors useful in inhibiting intake of alcohol. More specifically, these expression vectors work by inhibiting synthesis of catalase in brain tissue, or by destroying the product of catalase activity on ethyl alcohol.

### BACKGROUND AND PRIOR ART

While it is well known that an increase in blood acetaldehyde, generated mainly in the liver from ethanol by the action of the enzyme alcohol dehydrogenase which circulates in peripheral blood, produces a rejection of the consumption of alcohol (Mizoi et al., 1983; Peng et al.; 2007; Ocaranza et al. 2008; Rivera-Meta et al. 2010), it has also been shown that rodents will intracerebrally self administer acetaldehyde (Rodd et al., 2005; Amit et al., 1977; Brown et al. 1979), indicating a pleasant or reinforcing effect of this metabolite at the central nervous system level.

One of the questions in the alcoholism field is whether blood acetaldehyde, at the concentrations normally generated in the metabolism of ethanol (up to 50 micromolar) enters the brain via the blood-brain barrier. Studies indicate that since the capillaries of the blood-brain barrier do not present open pores, but rather tight junctions; in order to enter the brain, acetaldehyde must enter the capillary endothelial cells, richly endowed with high activity, high affinity aldehyde aldhydrogenase (ALDH2). Under normal conditions of ethanol metabolism, systemic acetaldehyde does not reach the cells in the central nervous system via the blood (Lindros and Hillbom, 1979; Peterson and Tabakoff, 1979; Eriksson 1977; Stowell et al. 1980). It has been estimated that only at blood concentrations of acetaldehyde higher than 100 micromolar (e.g., by injecting high doses of acetaldehyde), does it exceed the metabolic effect of the endothelial cells of the blood-brain barrier and enter the CNS (Tabakoff, Anderson et al. 1976).

Unlike the situation in the liver, there is no alcohol dehydrogenase in the brain to metabolize alcohol (Zimatkin and Deitrich 1977; Zimatkin et al. 2006), although acetaldehyde can be generated from ethanol by the action of catalase, and, to a lesser extent by cytochrome CYP2E1, as both these enzymes are present in the brain (Aragon et al. 1992; Zimatkin et al. 2006; Jamal et al. 2007). Catalase, although present in small amounts in all brain areas of the brain (Halliwell 2006; Moreno and cols, 1995), accounts for 70% of ethanol metabolized in the brain; while cytochrome CYP2E1 accounts for 20% (Zimatkin et al., 2006). Recently Sanchez-Catalan et al. (2008) reported the presence of CYP2E1 protein in the ventral tegmental area of the brain. In vivo studies (Zimatkin and Buben 2007) indicate that when infusing ethanol into rat brain ventricles, acetaldehyde can reach a concentration of 60 micromolar in the cerebrospinal fluid. The herbicide 3-amino-1,2,4 triazole (aminotriazole), a non specific inhibitor of catalase, lowered the levels of acetaldehyde. These studies suggest intracerebral generation of acetaldehyde in vivo, but the high concentrations of ethanol infused and the use of an inhibitor of catalase of low specificity such as that of aminotriazole do not allow one to conclude that catalase plays an important role in the consumption of alcohol.

Studies in which aminotriazole has been administered to subjects indicate that this drug reduces the voluntary consumption of alcohol in rats (Aragon and Amit 1992; Koechling et al. 1994; Gill et al. 1996; Tampier et al. 1995); however, it was also observed that aminotriazole inhibits the consumption of ethanol solutions and of food (Rotzinger et al. 1994, Tampier et al. 1995), showing that this inhibitor displays general effects in relation to the taste of a solution and also alters appetite.

Tampier et al. (1995) conclude that aminotriazole inhibits alcohol consumption by reducing the appetite for calories, and that this effect is related to its inhibitory effect upon catalase. Quertermont et al. in an exhaustive review of the effects of aminotriazole effects exerted by alcohol on the central nervous system conclude that there are no reliable data for determining whether cerebral catalase is or not involved in alcohol consumption. It should also be noted that the herbicide aminotriazole is a toxic compound and of short duration (http://pmep.cce.cornell.edu/profiles/herb-growthreg/24-d-butylate/amitrole/herb-prof-amitrole.html) which would require multiple administrations and, when systemically administered, inhibits catalase in several peripheral tissues, including the liver (Kanai et al. 1974). Rodd et al. (2005) demonstrated that rats will self-administer ethanol as well as acetaldehyde in the posterior ventral tegmental area (VIA), which contains dopaminergic neurons with projections toward the nucleus accumbens. Acetaldehyde (6×10⁻⁶ M) showed reinforcing effects at concentrations that are 1000 lower than those needed for alcohol (17×10⁻³ M). In such studies Rodd et al. concluded that aminotriazole did not inhibit the self-administration of ethanol.

In peripheral tissues such as the liver, catalase plays an important role in the detoxification of hydrogen peroxide generated in the dismutation of the superoxide radical; however, this is not the case in the brain (Halliwell, 2006) where, for the most part, other enzymes eliminate hydrogen peroxide (Turrens, 2003). These include the glutathione peroxidases and, recently, it has been suggested that the peroxiredoxins are even more important. (Rhee et al. 2005). These are enzymes that use cysteine and have a high affinity for hydrogen peroxide. If a highly specific inhibition of brain catalase could be achieved, this inhibition would not have toxic effects.

The development of specific gene-based inhibitors of catalase and its stereotactic administration in chosen areas of the brain would be valuable in determining if acetaldehyde is a mediator of reinforcing effects in the central nervous system. Prior to the invention presented herein, gene-based brain directed therapies were not known to inhibit chronic consumption of alcohol.

### SUMMARY OF THE INVENTION

The invention described herein relates to (1) the unexpected finding that gene based therapies are useful in inhibiting alcohol consumption in animals showing a natural predisposition towards developing alcoholism, (2) that the specific inhibition of brain catalase generates a reduction of alcohol intake that is virtually complete and, (3) lasts at least 50 days.

### BRIEF DESCRIPTION OF THE FIGURES

FIG. 1 shows the impact of the expression vectors of the invention in inhibition of catalase activity.

FIG. 2A presents a comparison of consumption and metabolism of alcohol by subject amounts which did or did not receive the expression vector of the invention.

FIG. 2B shows that food consumption was not affected by the therapy of the invention.

FIG. 2C demonstrates that water intake by subject animals was not impacted via the therapy of the invention.

### DETAILED DESCRIPTION OF THE PREFERRED EMBODIMENTS

Viral vectors with a high efficiency of gene transduction are generated in packaging cells, which generate the viral envelope proteins, which assemble DNA in their viral particles, and do not carry their own genes, but rather carry and transfer therapeutic or reporter genes. These genes may or may not be integrated in the genome of mammalian cells. Among those of the first type are the retroviral generated vectors, including but not limited to the murine leukemia virus (muLV) and the acquired immune deficiency lentiviral (HIV) vector.

The lentiviral vector presents characteristics that are highly favorable to deliver of therapeutic genes into cells of the central nervous system (Kitagawa et al. 2007), since it integrates into the genome of the host without activating oncogenes, and does not require that the nuclear membrane be temporally obliterated in the cell mitosis process. Lentiviral vectors have been used in numerous studies and are used in clinical studies (http://gemcris.od.nih.gov/Contents/GC_CT_RPT.asp?advsrch=true); however none of the studies show insertion of the vector via stereotactic injection into the brain.

### EXAMPLE 1

Firstly, lentiviral vectors were designed to code an RNA generated by the sequence

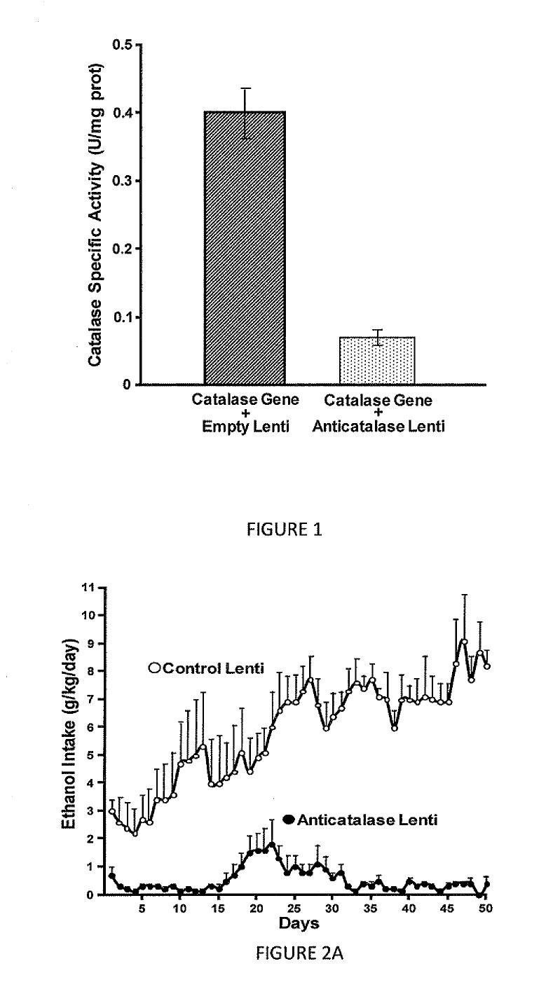

CCGGCCCTCTTATACCAGTTGGCAACTCGAGTTGCCAACTGGTATAAG AGGG (SEQ ID NO: 1) which inhibits the synthesis of catalase (anticatalase-Lenti). The methodologies for so doing are conventional to the skilled artisan. Briefly, a lentiviral vector is introduced into packaging cells (HEK 293T) along with three other plasmids that code the viral proteins Gag, Pol, Rev and VSV-G. The viral particles are harvested and concentrated from the supernatant of the culture by precipitation in the presence of polyethylene glycol-800. To demonstrate that catalase synthesis inhibitors were generated, human HEK 293 cells were transfected with a plasmid that codes rat catalase. Thereafter these cells were transduced with the lentiviral vector carrying the gene coding for the RNA anticatalase; which inhibited catalase activity by 80% (FIG. 1).

### EXAMPLE 2

Wistar rats, selected for their high alcohol consumption (Wistar UChB) were injected stereotactically in a dopaminergic area of the brain, and received 1 microliter (8×10⁴ viral particles) of anticatalase-Lenti or empty control virus (Control-Lenti). Four days after the injection the animals were placed in individual cages with two bottles; one containing 10% alcohol (10% ethanol v/v) and another one containing only water. Animals could select the tube from which they consume fluid, the volume of which was measured daily for 50 days.

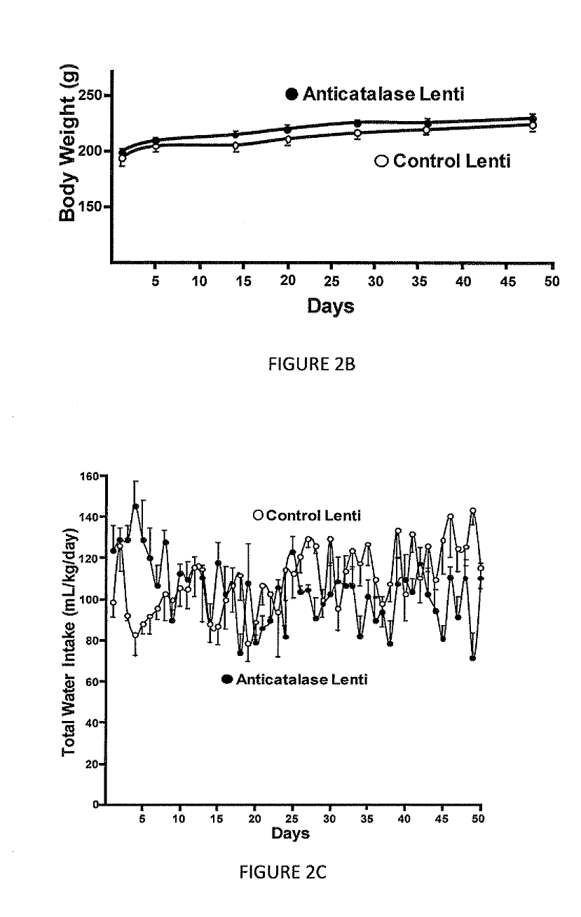

As can be observed in FIG. 2A, control animals (Control-Lenti) consumed and metabolized the equivalent of 500 ml of pure alcohol/70 kg and were dependent on alcohol (Ocaranza et al. 2008). The consumption levels were those expected for animals that have not received the administration of any compound (Quintanilla et al. 2006). However, animals to which an anticatalase vector was administered reduced their alcohol consumption by 95%, as seen at the end of the 50-day study. Since the anticatalase-Lenti remains permanently integrated in the brain of the animals, it is expected that this inhibition could last for the life of the animal.

FIG. 2B demonstrates that the weight of the animals was not affected by the administration of the anticatalase-Lenti vector; that is, it can be assumed that food intake did not change. FIG. 2C shows that total water consumption was not affected by the anticatalase-Lenti vector repeated studies generated the same results.

### EXAMPLE 3

In mammals, the nucleus accumbens is the sensor of a diverse number of mechanisms that are, in general pleasurable and desired by the animal. To determine the levels of dopamine in this area of the brain, a microdialysis probe was implanted to collect the fluid from the nucleus accumbens. It is well known that all addictive substances release dopamine into the nucleus accumbens. The studies shown above showed that the anticatalase-Lenti virus generated a state in which alcohol is no longer desirable to the animal, unlike the effect of an addictive substance; thus the administration of ethanol to these animals was not expected to liberate dopamine. It is, however, expected that if the anticatalase-Lenti virus treatment were specific for alcohol, a potent stimulant such as amphetamine would release high levels of dopamine in this area. Such was confirmed. In animals treated with Control-Lenti virus, administration of alcohol (1 g/kg) increased the dopamine levels in the nucleus accumbens by 52% (p<0.02), but such an increase was not present in animals administered anticatalase-Lenti virus (5%, N.S.). However, amphetamine administration increased the release of dopamine by 301% and 546% in animals treated with control-Lenti or anticatalase-Lenti virus (N.S. between both treatments), clearly indicating that the effect of the anticatalase-Lenti virus in inhibiting dopamine release in nucleus accumbens is specific for alcohol. The non-specific stimulation of dopamine release afforded by KCl infusion did not vary either when comparing both viral treatments (213 vs 277%; N.S.).

It will be clear to those of ordinary skill in the art that it is not only possible to inhibit the synthesis or activity of brain catalase to lower the generation or levels of brain acetaldehyde, but it is also possible to reduce alcohol consumption by a gene formulation that results in the generation of an enzyme that eliminates acetaldehyde, thus achieving the same effect.

Other features of the invention will be clear to the skilled artisan and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

### REFERENCES

Amit Z, Brown Z W, Rockman G E (1977) Possible involvement of acetaldehyde, norepinephrine and their tetrahydroisoquinoline derivatives in the regulation of ethanol self-administration. Drug Alcohol Depend 2:495-500.

Aragon C M, Amit Z (1992) The effect of 3-amino-1,2,4-triazole on voluntary ethanol consumption: evidence for brain catalase involvement in the mechanism of action. Neuropharmacology 31:709-712.

Aragon C M, Rogan F, Amit Z (1992) Ethanol metabolism in rat brain homogenates by a catalase-H2O2 system. Biochem Pharmacol 44:93-98.

Brown Z W, Amit Z, Rockman G E (1979) Intraventricular self-administration of acetaldehyde, but not ethanol, in naive laboratory rats. Psychopharmacology (Berl) 64:271-276.

Eriksson C J (1977) Acetaldehyde metabolism in vivo during ethanol oxidation. Adv Exp Med Biol 85A:319-341.

Gill K, Menez J F, Lucas D, Deitrich R A (1992) Enzymatic production of acetaldehyde from ethanol in rat brain tissue, Alcohol Clin Exp Res 16:910-915.

Halliwell B. Oxidative stress and neurodegeneration: where are we now? 2006. Journal of Neurochem. 97: 1634-1658.

Jamal M, Ameno K, Uekita I, Kumihashi M, Wang W, Ijiri I (2007) Catalase mediates acetaldehyde formation in the striatum of free-moving rats. Neurotoxicology 28:1245-1248.

Kanai Y, Sugimura T, Matsushima T, Kawamura A. (1974) Studies on in vivo degradation of rat hepatic catalase with or without modification by 3-amino-1,2,4-triazole. J Biol Chem. 249:6505-6511.

Kitagawa R, Miyachi S, Hanawa H, Takada M, Shimada T (2007) Differential characteristics of HIV-based versus SW-based lentiviral vector systems: Gene delivery to neurons and axonal transport of the expressed gene. Neuroscience Res. 57: 550-558.

Lindros K O, Hillbom M E (1979) Acetaldehyde in cerebrospinal fluid: its near-absence in ethanol-intoxicated alcoholics. Med Biol 57:246-247.

Moreno S, Mugnaiini E, Ceru M P. 1955. Immunocytochemical Localization of Catalase in the Central Nervous System of the Rat, J. Cytochem. Histochem, 43: 1253-1267, 1955.

Mizoi Y, Tatsuno Y, Adachi J, Kogame M, Fukunaga T, Fujiwara S, Hishida S, Ijiri I (1983) Alcohol sensitivity related to polymorphism of alcohol-metabolizing enzymes in Japanese. Pharmacol Biochem Behav 18 Suppl 1:127-133.

Ocaranza P, Quintanilla M E, Tampier L, Karahanian E, Sapag A, Israel Y (2008) Gene therapy reduces ethanol intake in an animal model of alcohol dependence. Alcohol Clin Exp Res 32:52-57.

Peng G S, Chen Y C, Tsao T P, Wang M F, Yin S J (2007) Pharmacokinetic and pharmacodynamic basis for partial protection against alcoholism in Asians, heterozygous for the variant ALDH2*2 gene allele Pharmacogenetics and Genomics 17:845-855.

Petersen D R, Tabakoff B (1979) Characterization of brain acetaldehyde oxidizing systems in the mouse. Drug Alcohol Depend 4:137-144.

Quertemont E, Tambour S, Tirelli E (2005) The role of acetaldehyde in the neurobehavioral effects of ethanol: a comprehensive review of animal studies. Prog Neurobiol 75:247-274.

Quintanilla M E, Israel Y, Sapag A, Tampier L (2006) The UChA and UChB rat lines: metabolic and genetic differences influencing ethanol intake. Addict Biol 11:310-323.

Rhee S. G., Chae H. Z. and Kim K. (2005) Peroxiredoxins: a historical overview and speculative preview of novel mechanisms and emerging concepts in cell signaling. Free Radic. Biol. Med. 38,1543-1552.

Rivera-Meza, Quintanilla M E, Tampier L, Mura C. Sapag A., Israel Y (2010). Mechanism of protection against alcoholism by an alcohol dehydrogenase polymorphism: development of an animal model. FASEB J. 24:266-274.

Rodd Z A, Bell R L, Zhang Y, Murphy J M, Goldstein A, Zaffaroni A, Li T K, McBride W J (2005) Regional heterogeneity for the intracranial self-administration of ethanol and acetaldehyde within the ventral tegmental area of alcohol-preferring (P) rats: involvement of dopamine and serotonin. Neuropsychopharmacology 30:330-338.

Rotzinger S, Smith B R, Amit Z (1994) Catalase inhibition attenuates the acquisition of ethanol and saccharin-quinine consumption in laboratory rats. Behav Pharmacol 5:203-209.

Sánchez-Catalán M J, Hipólito L, Guerri C, Granero L, Polache A (2008) Distribution and Differential Induction of CYP2E1 by Ethanol and Acetone in the Mesocorticolimbic System of Rat. Alcohol Alcohol 43: 401-407.

Tabakoff B, Anderson R A, Ritzmann R F (1976) Brain acetaldehyde after ethanol administration. Biochem Pharmacol 25:1305-1309.

Tampier L, Quintanilla M E, Mardones J (1995) Effects of aminotriazole on ethanol, water, and food intake and on brain catalase in UChA and UChB rats. Alcohol 12:341-344.

Turrens J F. Mitochondrial formation of reactive oxygen species. (2003) J Physiol 552-2: 335-344.

Zimatkin S M, Pronko S P, Vasiliou V, Gonzalez F J, Deitrich R A (2006) Enzymatic mechanisms of ethanol oxidation in the brain. Alcohol Clin Exp Res 30:1500-1505.

Zimatkin S M, Buben A L (2007) Ethanol oxidation in the living brain. Alcohol Alcohol 42:529-532.

1. A gene vector to be administered intracerebrally that inhibits the consumption of alcohol in mammals that show a marked appetite for its consumption.

2. A gene vector as the one claimed claim 1 that inhibits the consumption of alcohol for a minimum of 50 days following its single administration.

3. A gene vector that reduces the liberation of dopamine induced by an addictive substance, including ethanol.

4. A gene vector as the one claimed in claim 1 that inhibits the synthesis of catalase.

5. A gene vector as the one claimed in claim 2 that inhibits the synthesis of catalase.

6. A gene vector that promotes the elimination of the product generated in the action of catalase on ethyl alcohol

7. A gene vector that independently of its route of administration inhibits alcohol consumption for a minimum of 50 days following a single dose administration of such vector.
